(19) United States
(12) Patent Application Publication
(10) Pub. No.: US 2020/0276216 A1
(43) Pub. Date: Sep. 3, 2020

(54) INHIBITION OF GLYCOLYSIS WITH 2-DEOXY-D-GLUCOSE AND D-LACTIC ACID DIMER

(71) Applicant: Joel Steven Goldberg, Hillsborough, NC (US)

(72) Inventor: Joel Steven Goldberg, Hillsborough, NC (US)

(21) Appl. No.: 16/876,647

(22) Filed: May 18, 2020

(51) Int. Cl. A61K 31/7004; A61K 31/7016

(52) U.S. Cl. CPC A61K 31/7004; A61K 31/7016

(57) ABSTRACT

This invention targets the energy production that is need for viral replication and cancer cell growth by blocking glycolysis and by decreasing pyruvate that is available to undergo metabolism within the Krebs cycle with administration of a medication comprised of 2-deoxy-D-glucose (2DG) and D-lactic acid dimer (DLAD).

### FEDERALLY FUNDED REREARCH

None

### CROSS-REFERENCES TO RELATED APPLICATIONS

None

### BACKGROUND OF THE INVENTION

Hypermetabolic requirements for ATP that occur in cancer cell division and viral replication can be successful drug targets. Although there are three primary pathways to generate ATP (glycolysis, Krebs cycle, oxidative phosphorylation) the primary pathway for production of ATP in hypermetabolic states is glycolysis because the rate of ATP production is greatest. ATP can be generated from glycolysis in either aerobic or anaerobic environments however, regardless of the environment, glycolysis is the primary pathway to produce rapid quantities of ATP to satisfy consumption for energy needs.

In aerobic conditions and with functioning mitochondria the pyruvic acid end product of glycolysis can be shuttled to enter Krebs cycle and subsequent oxidative phosphorylation to produce approximately 32 ATP per 1 molecule of glucose. In anaerobic conditions the final product of glycolysis is lactic acid. Either glycolytic process produces ATP at a far greater rate than oxidative phosphorylation.

Glycolysis in Viral Infections

Recent evident in Tomato bushy stunt virus, an RNA virus, suggests that ATP is generated by glycolysis and fermentation in viral replication complexes contiguous with the cytosol after viral infection.[1] The glycolytic pathway may be the more significant of the two pathways. It therefore seems reasonable to consider pharmacologic therapies that interrupt glycolysis as antiviral medications.

Most present day antiviral medications target enzyme inhibitors. For RNA viruses, the targeted inhibitory enzymes are associated with viral adsorption, viral fusion, reverse transcription, integration, transcription, and protein degradation.[2] No clinically available antiviral medications are available that address the energy production of viral replication, yet enormous amount of ATP are required for this process to continue.

Glycolysis in Cancer

During the last century, Otto Warburg demonstrated that the glucose requirement for cancer cells far exceeded the requirement for non-cancer cells and this has been named the Warburg effect. One proof of concept of this effect has been the localization of cancer through administration of 2-fluoro2-deoxy-D-glucose and imaging with a PET scanner.

2-Dexoxy-D-Glucose (2DG)

2GD is an analog of glucose that inhibits glycolysis by primarily by competitively binding to hexokinase the first step in glycolysis. 2DG has known antiviral and tumoricidal properties that have been demonstrated in multiple in vitro trials and animal trials.[3] 2DG is a small molecule that is easy and cheap to synthesis. It can be administered intravenously or orally with an estimated maximum approximate dose of 60 mg/kg/day before the side effects of fatigue, dizziness, hyperglycemia, hypoglycemia, sweating, nausea, QTc prolongation, and gastrointestinal bleeding become clinically significant. [4] 2DG can be administered orally with the half-life ranging from 4.5 to 8.2 hours depending upon the dose ranges.[5] It is presumed that most non-viral infected cells or non-cancer cells that do not require the high rate of production ATP can adapt to disruption in glycolysis because other pathways to produce ATP exists through oxidation of free fatty acids and amino acids that can be processed in the Krebs cycle and oxidative phosphorylation. In theory, but not clinically apparent, the most likely cells to be affected by 2DG are red blood cells since they do not have mitochondria and are unable to process fuel through the Krebs cycle and oxidative phosphorylation

D-Lactic Acid Dimer (DLAD)

D-lactic acid dimer is a small molecule that can be easily and simply synthesized through the ester condensation of D-lactic acid. It sequesters or traps L-lactate.[6] This is reaction occurs spontaneously and is non-enzymatic.[7] Being able to target an important biologic intermediary as L-lactate without enzyme inhibition suggests that more complex changes in the host would need to take place to develop resistance. That is, resistance to an infective agent or tumor can occur with small changes in the inhibited enzyme either through epigenetic or changes in the DNA. However, a medication that does not depend upon inactivity of an enzyme may be less susceptible to viral or tumor resistance. Nothing is known about the bioavailability DLAD. The mechanism of sequestration of L-lactate by DLAD is believed to occur from the stereo complex formed when DLAD reacts with L-lactate.[7] The proof of the reaction has been demonstrated by HPLC and by direct observation of a precipitate formed when the two molecules come together.[7]

The mechanism of action of DLAD in biological systems is exclusive of its ability to lower pH, is dose dependent in vitro, and selective for cancer cells.[8] DLAD has never been shown to have antiviral activity, but its presumed mechanism of action is inhibition of glycolysis. Sequestration of L-lactate may impair glycolysis by interrupting the buffering of end product hydrogen ions generation in the cytosol. In addition, sequestration of L- lactate may change the equilibrium concentration of pyruvate conversion to lactic which is a reversible reaction catalyzed by various isoforms of LDH. Therefore, sequestration of lactate will act as a sink driving the reaction toward completion and in the process decreases the quantities of pyruvate that can be used as fuel to enter Krebs cycle via acetyl-coA and subsequently oxidation phosphorylation to produce ATP.

There is good evidence that glycolysis is the primary pathway for generation of ATP in hypermetabolic processes. Thus, this invention proposes that a combination of 2DG and DLAD will inhibit glycolysis preferentially in cancer cells and virally infected cells.

### DESCRIPTION OF THE DRAWINGS

None

### DETAIL DESCRIPTION OF THE INVENTION

This invention is a method to treat hypermetabolic states that occur in many diseases such as viral infections and cancer with a medication comprised of 2DG and DLAD which will interrupt glycolysis, the principle the source of ATP required for viral replication and tumor cell growth. The rate of production of ATP from glycolysis compare to Krebs cycle and oxidation phosphorylation is greater even though the utilization for glucose is less efficient on a molar basis. Without knowing the kinetics of each reaction, perhaps this difference in rate of ATP production is related to the number of reactions and transportation of intermediates from the cytosol to the mitochondria since: a) Glycolysis is 4 unique reactions that produces 2 net ATP, b) Krebs cycle is 9 unique reactions that produces 2 net ATP, and there is transportation time needed for pyruvate to enter the mitochondria from the cytosol, c) Oxidative phosphorylation is an unknown number of reactions and produces about 30 net ATP in the mitochondria.

No present antiviral or chemotherapeutic agents target the energy supply needed for viral replication or tumor cell growth. Most antiviral medications target various enzyme systems and many chemotherapeutic agents also target enzyme systems. Targeting enzyme systems can lead to resistance because changes in enzyme expression or structure can occur based on small changes in the DNA sequence for the enzyme or changes in epigenetics.

2DG is a known antiviral. It can be administered orally or intravenously. DLAD is a known tumoricidal agent in vitro and in vivo in mice. Its bioavailability is unknown. Its mechanism of action is non-enzymatic spontaneous sequestration of L-lactate through formation of a stereo complex. Its metabolism is presumed to be ester hydrolysis to D-lactic acid that can be excreted in the urine. Its toxicity is unknown, but judging from the elution of polylactide stents which contain D-lactic acid the toxicity is probably low.

The combined therapy of 2DG and DLAD will interrupt glycolysis in two different ways. As an analog of glucose, 2DG will primarily competitively inhibit hexokinase the first enzyme in glycolysis that converts glucose to glucose 6-phosphate. DLAD will interrupt glycolysis by sequestration of L-lactate that will decrease the buffering of hydrogen ions produced as an end product of this process. Sequestration of L-Lactate will also decrease pyruvate available to be shuttled into the Krebs cycle.

### Benefits to Society

Pharmacologic targeting of the energy production for viral replication and for cancer cell growth may have profound applications for the treatment of viral infections and cancer.

### REFERENCES

1. Nagy, P. D. and W. Lin, Taking over Cellular Energy-Metabolism for TBSV Replication: The High ATP Requirement of an RNA Virus within the Viral Replication Organelle. Viruses, 2020. 12(1).
2. De Clercq, E., Antivirals and antiviral strategies. Nat Rev Microbiol, 2004. 2(9): p. 704-20.
3. Kilbourne, E. D., Inhibition of influenza virus multiplication with a glucose antimetabolite (2-deoxy-D-glucose). Nature, 1959. 183(4656): p. 271-2.
4. Raez, L. E., et al., A phase I dose-escalation trial of 2-deoxy-D-glucose alone or combined with docetaxel in patients with advanced solid tumors. Cancer Chemother Pharmacol, 2013. 71(2): p. 523-30.
5. Stein, M., et al., Targeting tumor metabolism with 2-deoxyglucose in patients with castrate-resistant prostate cancer and advanced malignancies. Prostate, 2010. 70(13): p. 1388-94.
6. Goldberg, J. S., Gooden, D. M., SYNTHESIS AND IN VITRO ACTIVITY OF D-LACTIC ACID OLIGOMERS, US 2018/0273464 A1, USPTO.
7. Goldberg, J. S., Weinberg, J. B., USE OF POLYMER D-LACTIC ACID (PDLA) OR EQUIVALENTS THEREOF TO INHIBIT GROWTH OF CANCER CELLS AND DIAGNOSE CANCERS, U.S. Pat. No. 9,382,376 B2, USPTO.
8. Goldberg, J. S., Weinberg, J. B., LOCAL APPLICATION OF D-LACTIC ACID DIMER IS SELECTIVELY CYTOTOXIC WHEN APPLIED TO CANCER CELLS, U.S. Pat. No. 10,034,895 B2, USPTO.

Having described my invention, I claim:

1. A method to inhibit the glycolytic process within cells in a subject by administering to said subject a composition comprising 2-deoxy-D-glucose and D-lactic acid dimer.

2. A method of treating cancer in a subject comprising administering to said subject a composition comprising 2-deoxy-D-glucose and D-lactic acid dimer.

3. The method of claim 2 where a prodrug of D-lactic acid dimer is administered to said subject.

4. A method of treating viral infection in a subject comprising administering to said subject a composition comprising 2-deoxy-D-glucose and D-lactic acid dimer.

5. The method of claim 4 where a prodrug of D-lactic acid dimer is administered to said subject.
